(19) United States
(12) Patent Application Publication
(10) Pub. No.: US 2023/0002439 A1
(43) Pub. Date: Jan. 5, 2023

(54) CRYSTALLINE FORMS OF VAMOROLONE

(71) Applicant: ReveraGen BioPharma, Inc., Rockville, MD (US)

(72) Inventors: John MCCALL, Boca Grande, FL (US); Jesse DAMSKER, Alexandria, VA (US)

(21) Appl. No.: 17/806,322

(22) Filed: Jun. 10, 2022

Related U.S. Application Data
(60) Provisional application No. 63/209,459, filed on Jun. 11, 2021.

(51) Int. Cl. C07J 5/00

(52) U.S. Cl. CPC C07J 5/0076; C07B 2200/13

(57) ABSTRACT

Provided are certain polymorphic forms of vamorolone as well as pharmaceutical compositions and methods for their use.

This application claims the benefit of priority of U.S. Provisional Patent Application Ser. No. 63/209,459 filed Jun. 11, 2021, the disclosure of which is incorporated by reference in its entirety for all purposes.

This disclosure relates to crystalline forms of vamorolone, related compositions, and related methods of formation and use.

Solids exist in either amorphous or crystalline forms. In the case of crystalline forms, molecules are positioned in three-dimensional lattice sites. When a compound recrystallizes from a solution or slurry, it may crystallize with different spatial lattice arrangements, and the different crystalline forms are sometimes referred to as “polymorphs.” The different crystalline forms of a given substance may differ from each other with respect to one or more chemical properties (e.g., dissolution rate, solubility), biological properties (e.g., bioavailability, pharmacokinetics), and/or physical properties (e.g., mechanical strength, compaction behavior, flow properties, particle size, shape, melting point, degree of hydration or solvation, caking tendency, compatibility with excipients). The variation in properties among different crystalline forms usually means that one crystalline form may be more useful than other forms.

Vamorolone is a synthetic glucocorticoid corticosteroid, also known as VB-15, VBP-15, 16α-methyl-9,11-dehydroprednisolone, or 17α,21-dihydroxy-16α-methylpregna-1,4,9(11)-triene-3,20-dione:

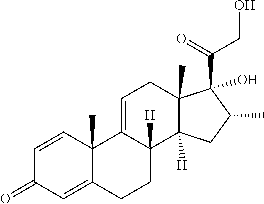

Pre-clinical data have shown that vamorolone binds potently to the glucocorticoid receptor and has anti-inflammatory effects similar to traditional glucocorticoid drugs. Testing multiple mouse models of inflammatory states has shown efficacy similar to prednisolone and dramatically improves side effect profiles, including loss of growth stunting and loss of bone fragility.

Because vamorolone exhibits several advantageous therapeutic properties, improved forms of the compound are desired, particularly regarding enhanced solubility, bioavailability, ease of synthesis, ability to be readily formulated, and/or physical stability. Thus, there is a need for improved crystalline forms of vamorolone and methods for preparing the different forms.

Provided is a compound which is vamorolone Form II, vamorolone Form III, vamorolone Form IV, vamorolone Form V, vamorolone Form VI, vamorolone Form VII, or vamorolone Form VIII. Also provided are mixtures of one or more of those polymorphic forms of vamorolone. Also provided are pharmaceutical compositions comprising one or more polymorphic forms of vamorolone and at least one pharmaceutically acceptable excipient. Also provided are methods for using the polymorphic forms of vamorolone and/or pharmaceutical compositions thereof. Also provided are processes for preparing the various polymorphic forms of vamorolone and products of those processes.

These and other aspects of the invention disclosed herein will be set forth in greater detail as the patent disclosure proceeds.

### BRIEF DESCRIPTION OF THE DRAWINGS

FIGS. 1 and 2 show a pXRD pattern of vamorolone Form II prepared by heating at 205° C. on the hot-stage (upper) compared to Form I vamorolone (lower).

FIGS. 3 and 4 show a pXRD pattern of vamorolone Form III (upper) compared to Form I vamorolone (lower).

FIGS. 5 and 6 show a pXRD pattern of vamorolone Form IV (upper) compared to Form I vamorolone (lower).

FIG. 7 shows a TGA profile of vamorolone Form IV.

FIG. 8 shows a pXRD pattern of vamorolone Form V (upper) compared to Form I vamorolone (lower).

FIG. 9 shows a TGA profile of vamorolone Form V.

FIG. 10 shows a DSC profile of vamorolone Form V.

FIGS. 11 and 12 show a pXRD pattern of vamorolone Form VI (upper) compared to Form I vamorolone (lower).

FIG. 13 shows a pXRD pattern of vamorolone Form VII (upper) compared to Form I vamorolone (lower).

FIG. 14 shows a TGA profile of vamorolone Form VII.

FIG. 15 shows a DSC profile of vamorolone Form VII.

FIGS. 16 and 17 show a pXRD pattern of vamorolone Form VIII (upper) compared to Form I vamorolone (lower).

FIG. 18 shows a TGA profile and a DSC profile of vamorolone Form VIII.

FIG. 19 shows a pXRD pattern of a mixture of Forms I, III-VIII after slurry in water (upper) compared to Form I vamorolone (lower).

FIG. 20 is a summary of polymorphs obtained from solvent crystallizations (as in the 96-well plate.)

### DETAILED DESCRIPTION

As used in the present specification, the following words and phrases are generally intended to have the meanings as set forth below, except to the extent that the context in which they are used indicates otherwise.

VAMOROLONE: As used herein, “vamorolone” refers to 17α,21-dihydroxy-16α-methylpregna-1,4,9(11)-triene-3,20-dione (also known as VBP15) and has the structure:

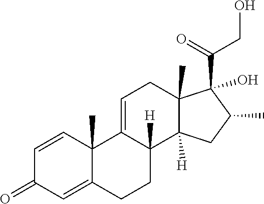

POLYMORPHS AND POLYMORPHIC FORMS: Vamorolone can exist in various polymorphic forms. As used herein, the terms “polymorphs” and “polymorphic forms” and related terms herein refer to crystalline forms of the same molecule. Different polymorphs may have different physical properties such as, for example, melting temperatures, heats of fusion, solubilities, dissolution rates, and/or vibrational spectra because of the arrangement or conformation of the molecules in the crystal lattice. The differences in physical properties exhibited by polymorphs affect pharmaceutical parameters such as storage stability, compressibility and density (important in formulation and product manufacturing), and dissolution rates (an important factor in bioavailability). Differences in stability can also result from changes in chemical reactivity (e.g., differential oxidation, such that a dosage form discolors more rapidly when comprised of one polymorph than when comprised of another polymorph) or mechanical property (e.g., tablets crumble on storage as a kinetically favored polymorph converts to thermodynamically more stable polymorph) or both (e.g., tablets of one polymorph are more susceptible to breakdown at high humidity). As a result of solubility/dissolution differences, in the extreme case, some polymorphic transitions may result in a lack of potency or, at the other extreme, toxicity. In addition, the physical properties of the crystal may be important in processing. For example, one polymorph might be more likely to form solvates or might be difficult to filter and wash free of impurities (i.e., particle shape and size distribution might differ between polymorphs).

Polymorphs of a molecule can be obtained by several methods, as known in the art. Such methods include, but are not limited to, melt recrystallization, melt cooling, solvent recrystallization, desolvation, rapid evaporation, rapid cooling, slow cooling, vapor diffusion, and sublimation.

Techniques for characterizing polymorphs include, but are not limited to, differential scanning calorimetry (DSC), X-ray powder diffractometry (XRPD), single-crystal X-ray diffractometry, vibrational spectroscopy, e.g., IR and Raman spectroscopy, solid-state NMR, hot stage optical microscopy, scanning electron microscopy (SEM), electron crystallography and quantitative analysis, particle size analysis (PSA), surface area analysis, solubility studies, and dissolution studies.

To “characterize” a solid form of a compound, one may, for example, collect XRPD data on solid forms of the compound and compare the XRPD peaks of the forms. For example, when only three solid forms, e.g., Forms X and Y and Material N, are compared. The Form X pattern shows a peak at an angle where no peaks appear in the Form Y or Material N pattern, then that peak, for that compound, distinguishes Form X from Form Y and Material N and further acts to characterize Form X. The collection of peaks that distinguish, e.g., Form X from the other known forms is a collection of peaks that characterize Form X. Those of ordinary skill in the art will recognize that there are often multiple ways, including multiple ways using the same analytical technique, to characterize solid forms. Additional peaks could also be used, but are not necessary, to characterize the form up to and including an entire diffraction pattern. Although all the peaks within an entire XRPD pattern may be used to characterize such a form, a subset of that data may be, and typically is, used to characterize the form.

An XRPD pattern is an x-y graph with a diffraction angle (typically ° 2θ) on the x-axis and intensity on the y-axis. The peaks within this pattern may be used to characterize a crystalline solid form. As with any data measurement, there is variability in XRPD data. The data are often represented solely by the diffraction angle of the peaks rather than including the intensity of the peaks because peak intensity can be particularly sensitive to sample preparation (for example, particle size, moisture content, solvent content, and preferred orientation effects influence the sensitivity), so samples of the same material prepared under different conditions may yield slightly different patterns; this variability is usually greater than the variability in diffraction angles. Diffraction angle variability may also be sensitive to sample preparation. Other sources of variability come from instrument parameters and processing of the raw X-ray data: different X-ray instruments operate using different parameters. These may lead to slightly different XRPD patterns from the same solid form, and similarly, different software packages process X-ray data differently. This also leads to variability. These and other sources of variability are known to those of ordinary skill in the pharmaceutical arts. Due to such sources of variability, it is usual to assign a variability of ±0.2° 2θ to diffraction angles in XRPD patterns.

ABOUT: As used herein, the term “about” is intended to qualify the numerical values it modifies, denoting such a value as a variable within a margin of error. When no particular margin of error, such as a standard deviation to a mean value given in a chart or table of data, is recited, the term “about” should be understood to mean that range that would encompass the recited value and the range which would be included by rounding up or down to that figure as well, taking into account significant figures.

ADMINISTERING: As used herein, “administering” means to provide a compound or other therapy, remedy, or treatment such that an individual internalizes a compound.

DISEASE: As used herein, the term “disease” is intended to be generally synonymous and is used interchangeably with the terms “disorder” and “condition” (as in medical condition), in that all reflect an abnormal condition of the human or animal body or of one of its parts that impairs normal functioning, is typically manifested by distinguishing signs and symptoms and causes the human or animal to have a reduced duration or quality of life.

IN NEED OF TREATMENT and IN NEED THEREOF: As used herein, “in need of treatment” and “in need thereof” when referring to treatment are used interchangeably to mean a judgment made by a caregiver (e.g., physician, nurse, nurse practitioner, etc. in the case of humans; veterinarian in the case of animals, including non-human mammals) that an individual or animal requires or will benefit from treatment. This judgment is made based on a variety of factors in the realm of a caregiver's expertise, but that includes the knowledge that the individual or animal is ill or will become ill as the result of a disease, condition, or disorder that is treatable by the compounds of the invention. Accordingly, the compounds of the invention can be used in a protective or preventive manner; or compounds of the invention can alleviate, inhibit or ameliorate the disease, condition, or disorder.

NF-κB-MEDIATED DISEASE: As used herein, the term “NF-κB-mediated disease” refers to a disease having a significant and pathologic inflammatory component that can be addressed by inhibition of NF-κB. The disease may be completely or partially mediated by modulating the activity or amount of NF-κB. In particular, the disease is one in which modulation of NF-κB results in some effect on the underlying disease, e.g., administration of NF-κB modulator results in some improvement in at least some of the patients being treated. The term “NF-κB-mediated disease” also refers to the following diseases, even though the compounds disclosed herein exert their effects through biological pathways and/or processes other than NF-κB: muscular dystrophy, arthritis, traumatic brain injury, spinal cord injury, sepsis, rheumatic disease, cancer atherosclerosis, type 1 diabetes, type 2 diabetes, leptospiriosis renal disease, glaucoma, retinal disease, ageing, headache, pain, complex regional pain syndrome, cardiac hypertrophy, muscle wasting, catabolic disorders, obesity, fetal growth retardation, hypercholesterolemia, heart disease, chronic heart failure, ischemia/reperfusion, stroke, cerebral aneurysm, angina pectoris, pulmonary disease, cystic fibrosis, acid-induced lung injury, pulmonary hypertension, asthma, chronic obstructive pulmonary disease, Sjogren's syndrome, hyaline membrane disease, kidney disease, glomerular disease, alcoholic liver disease, gut diseases, peritoneal endometriosis, skin diseases, nasal sinusitis, mesothelioma, anhidrotic ecodermal dysplasia-ID, behcet's disease, incontinentia pigmenti, tuberculosis, asthma, crohn's disease, colitis, ocular allergy, appendicitis, paget's disease, pancreatitis, periodonitis, endometriosis, inflammatory bowel disease, inflammatory lung disease, silica-induced diseases, sleep apnea, AIDS, HIV-1, autoimmune diseases, antiphospholipid syndrome, lupus, lupus nephritis, familial mediterranean fever, hereditary periodic fever syndrome, psychosocial stress diseases, neuropathological diseases, familial amyloidotic polyneuropathy, inflammatory neuropathy, parkinson's disease, multiple sclerosis, alzheimer's disease, amyotropic lateral sclerosis, huntington's disease, cataracts, and hearing loss.

PHARMACEUTICAL COMPOSITION: As used here, “pharmaceutical composition” means a composition comprising at least one active ingredient, such as vamorolone or a polymorphic form thereof, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

PURE: As used herein, the term “pure” means about 90-100%, preferably 95-100%, more preferably 98-100% (wt/wt) or 99-100% (wt/wt) pure compound; e.g., less than about 10%, less than about 5%, less than about 2% or less than about 1% impurity is present. Such impurities include, e.g., degradation products, oxidized products, epimers, solvents, and/or other undesirable impurities.

RANGES: When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons since carbons come in integer units. Compare, by way of example, the range “from 1 to 3 μM (micromolar),” which is intended to include 1 μM, 3 μM, and everything in between, to any number of significant figures (e.g., 1.255 μM, 2.1 μM, 2.9999 μM, etc.).

ROOM TEMPERATURE: As used herein, the term “room temperature” refers to a temperature of 68 to 86 F.

THERAPEUTICALLY ACCEPTABLE: As used herein, the term “therapeutically acceptable” refers to those compounds (or salts, prodrugs, tautomers, zwitterionic forms, etc.) suitable for use in contact with the tissues of patients without undue toxicity, irritation, and allergic response, are commensurate with a reasonable benefit/risk ratio and are effective for their intended use.

THERAPEUTICALLY EFFECTIVE: As used herein, the phrase “therapeutically effective” is intended to qualify the amount of active ingredients used in the treatment of a disease or disorder. This amount will achieve the goal of reducing or eliminating the disease or disorder.

TREATMENT: As used herein, “treating,” “treatment,” and the like means ameliorating a disease to reduce or eliminate its cause, its progression, its severity, or one or more of its symptoms, or otherwise beneficially alter the disease in a subject. Treatment may also be preemptive, i.e., it may include prophylaxis of disease in a subject exposed to or at risk for the disease. Prevention of a disease may involve complete protection from disease, such as prevention of infection with a pathogen, or may involve prevention of disease progression, for example, from prediabetes to diabetes. For example, prevention of a disease may not mean complete foreclosure of any effect related to the disease at any level. Instead, it may mean preventing the symptoms of a disease to a clinically significant or detectable level. Prevention of diseases may also mean prevention of the progression of a disease to a later stage of the disease.

Throughout this specification, unless the context requires otherwise, the word “comprise,” or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e., one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all the steps, features, compositions, and compounds referred to or indicated in this specification, individually or collectively, and all combinations or any two or more of the steps or features unless specifically stated otherwise.

The present invention(s) is not limited in scope by the specific embodiments described herein, which are intended for exemplification only. Functionally-equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination.

Provided is a polymorphic form of vamorolone chosen from vamorolone Form II, vamorolone Form III, vamorolone Form IV, vamorolone Form V, vamorolone Form VI, vamorolone Form VII, and vamorolone Form VIII. Also provided are mixtures of one or more of those polymorphic forms of vamorolone.

In some embodiments, the polymorphic form of vamorolone has a chemical purity of at least about 95%, at least about 96%, at least about 97%, at least about 98%, at least about 99%, or at least about 99.5% by high-performance liquid chromatography.

In some embodiments, the polymorphic form of vamorolone has total chemical impurities of not more than about 0.5%, not more than about 0.4%, not more than about 0.3%, not more than about 0.2%, or not more than about 0.1%, by high-performance liquid chromatography.

In some embodiments, the polymorphic form of vamorolone has not more than about 0.5%, not more than about 0.4%, not more than about 0.3%, not more than about 0.2%, or not more than about 0.1%, by high-performance liquid chromatography of an epoxide having the formula:

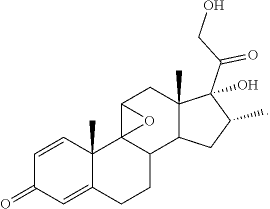 [SMILES: C[C@@H]1CC2C3CCC4=CC(=O)C=C[C@]4(C)C34OC4C[C@]2(C)[C@@]1(O)C(=O)CO]

In some embodiments, the polymorphic form of vamorolone has not more than about 0.5%, not more than about 0.4%, not more than about 0.3%, not more than about 0.2%, or not more than about 0.1%, by high-performance liquid chromatography of a ketone having the formula:

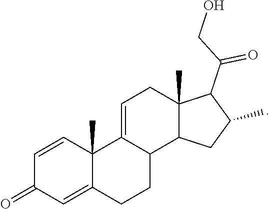 [SMILES: C[C@@H]1CC2C3CCC4=CC(=O)C=C[C@]4(C)C3=CC[C@]2(C)C1C(=O)CO]

In some embodiments, the polymorphic form of vamorolone has not more than about 0.5%, not more than about 0.4%, not more than about 0.3%, not more than about 0.2%, or not more than about 0.1%, by high-performance liquid chromatography of a diastereomeric compound having the formula:

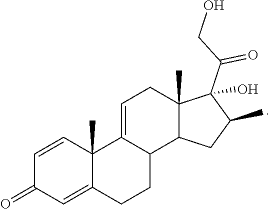 [SMILES: C[C@H]1CC2C3CCC4=CC(=O)C=C[C@]4(C)C3=CC[C@]2(C)[C@@]1(O)C(=O)CO]

In some embodiments, the polymorphic form of vamorolone has not more than about 0.5%, not more than about 0.4%, or not more than about 0.3%, by high-performance liquid chromatography of an acetate having the formula:

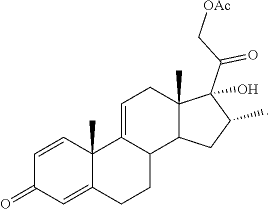 [SMILES: CC(=O)OCC(=O)[C@@]1(O)[C@H](C)CC2C3CCC4=CC(=O)C=C[C@]4(C)C3=CC[C@@]21C]

The acetate impurity is also referred to as the impurity having a relative retention time of 1.38-1.40.

### Vamorolone Form II

Provided is a compound which is vamorolone Form II.

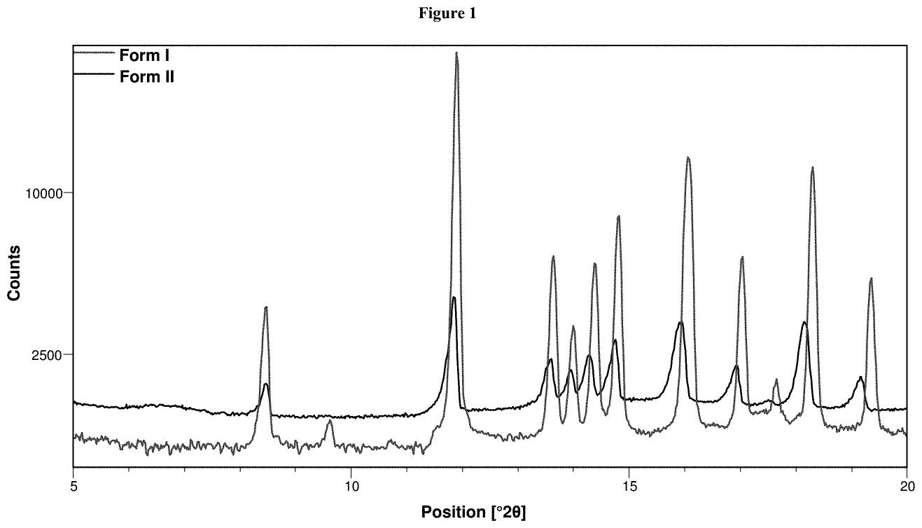

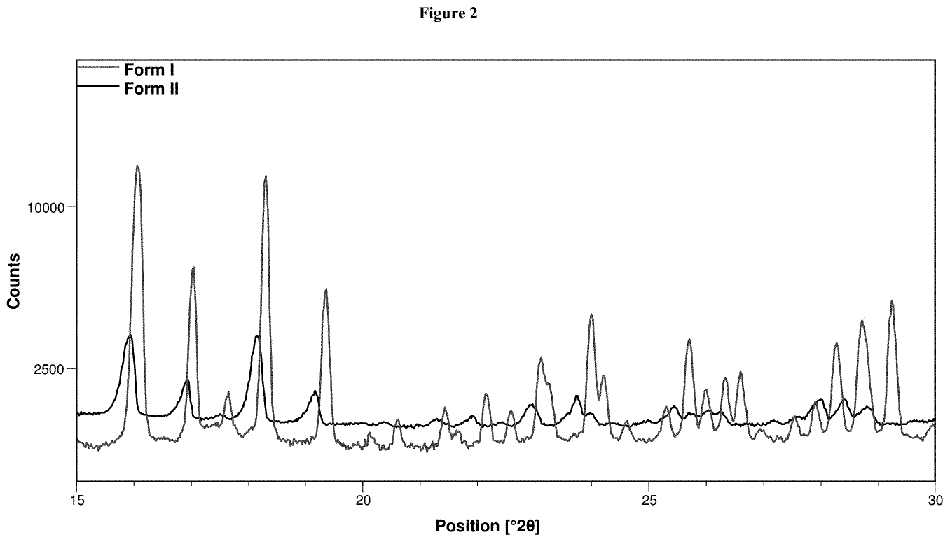

In some embodiments, vamorolone Form II is characterized by an X-ray powder diffraction pattern comprising peaks, in terms of ° 2θ, at about 11.8, about 15.9, and about 18.2 with radiation Cu Kα. In some embodiments, vamorolone Form II is characterized by an X-ray powder diffraction pattern comprising peaks, in terms of ° 2θ, at about 11.8, about 13.6, about 14.3, about 14.7, about 15.9, about 17.0, and about 18.2 with radiation Cu Kα. In some embodiments, vamorolone Form II is characterized by an X-ray powder diffraction pattern substantially, as shown in FIG. 1 or 2.

### Vamorolone Form III

Provided is a compound which is vamorolone Form III.

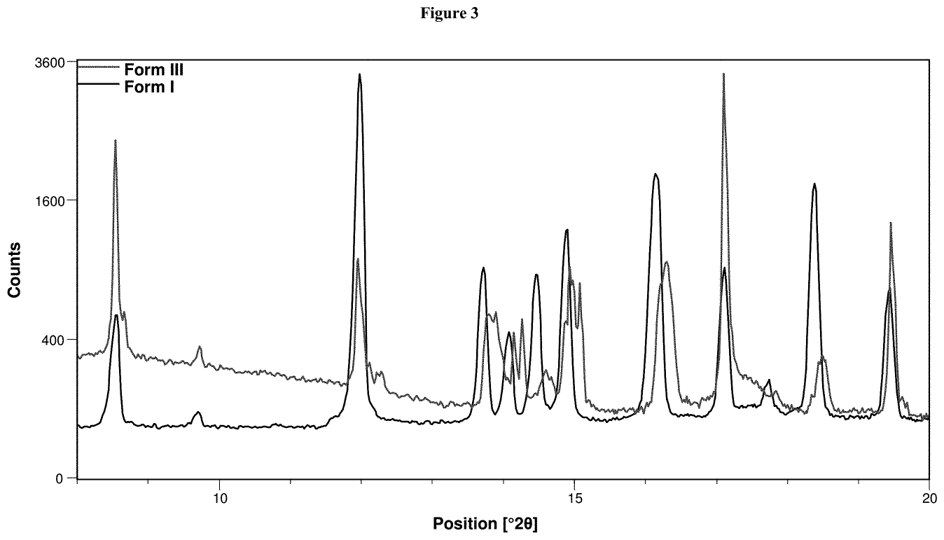

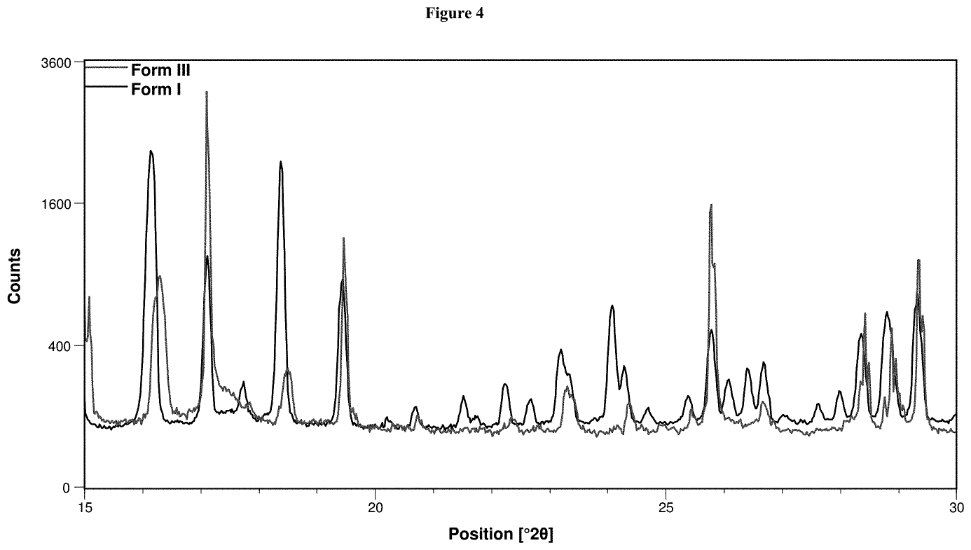

In some embodiments, vamorolone Form III is characterized by an X-ray powder diffraction pattern comprising peaks, in terms of ° 2θ, at about 19.4, about 25.8, and 29.3 with radiation Cu Kα. In some embodiments, vamorolone Form III is characterized by an X-ray powder diffraction pattern comprising peaks, in terms of ° 2θ, at about 9.5, about 17.2, about 19.4, about 25.8, and 29.3 with radiation Cu Kα. In some embodiments, vamorolone Form III is characterized by an X-ray powder diffraction pattern substantially, as shown in FIG. 3 or 4.

In some embodiments, vamorolone Form III is prepared by a process comprising the step of slowly concentrating by evaporation at room temperature a solution of vamorolone in a solvent and isolating the resulting crystals. In some embodiments, the solvent is dichloromethane or a mixture of dichloromethane/heptane.

### Vamorolone Form IV

Provided is a compound which is vamorolone Form IV.

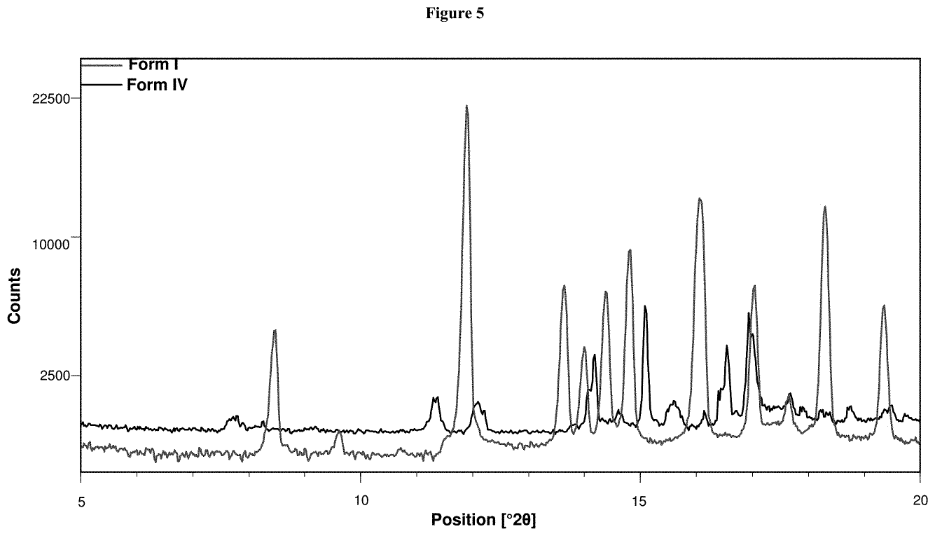

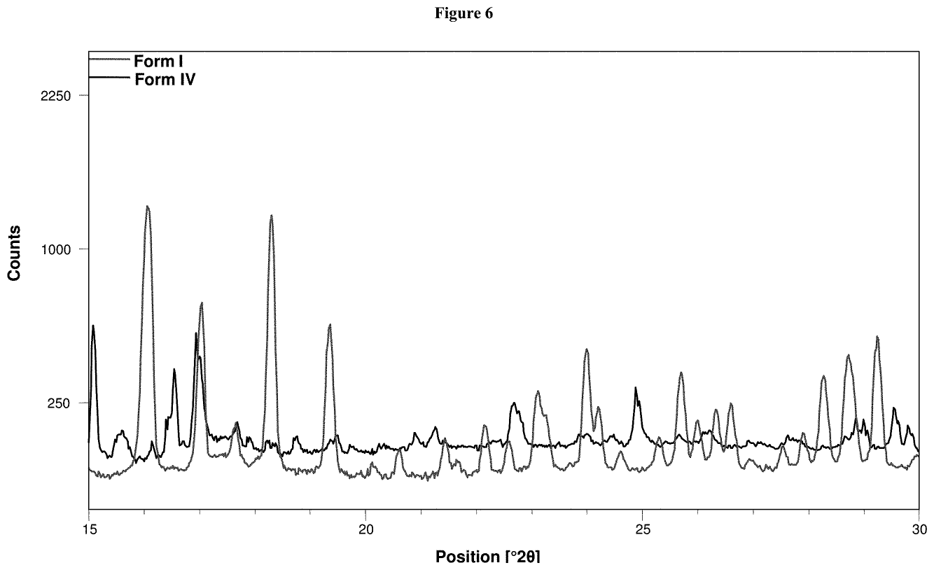

In some embodiments, vamorolone Form IV is characterized by an X-ray powder diffraction pattern comprising peaks, in terms of ° 2θ, at about 14.2, about 15.1, about 16.8, about 17.0, and about 24.8 with radiation Cu Kα. In some embodiments, vamorolone Form IV is characterized by an X-ray powder diffraction pattern comprising peaks, in terms of ° 2θ, at about 14.2, about 15.1, about 16.5, about 16.8, about 17.0, and about 24.8 with radiation Cu Kα. In some embodiments, vamorolone Form IV is characterized by an X-ray powder diffraction pattern substantially, as shown in FIG. 5 or 6.

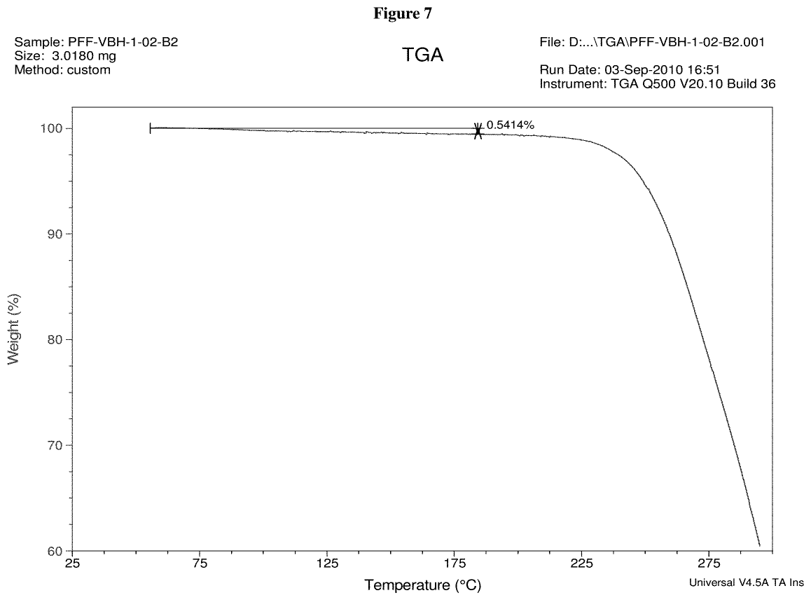

In some embodiments, vamorolone Form IV is characterized by a thermogravimetric analysis profile showing less than about 0.5% weight loss below about 200° C. In some embodiments, vamorolone Form IV is characterized by a thermogravimetric analysis profile substantially, as shown in FIG. 7.

In some embodiments, vamorolone Form IV is prepared by a process comprising the step of slowly concentrating by evaporation at room temperature a solution of vamorolone in a solvent and isolating the resulting crystals. In some embodiments, the solvent is acetone or a mixture of ethyl acetate/methanol.

### Vamorolone Form V

Provided is a compound which is vamorolone Form V.

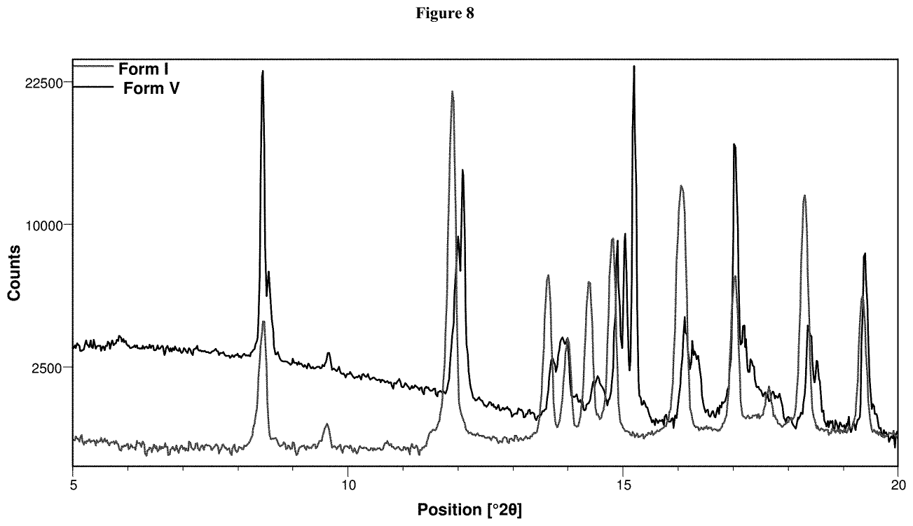

In some embodiments, vamorolone Form V is characterized by an X-ray powder diffraction pattern comprising peaks in terms of ° 2θ, about 12.1, about 14.8, about 15.0, and about 15.2 with radiation Cu Kα. In some embodiments, vamorolone Form V is characterized by an X-ray powder diffraction pattern comprising peaks, in terms of ° 2θ, at about 8.3, about 11.9, about 12.1, about 14.8, about 15.0, and about 15.2 with radiation Cu Kα. In some embodiments, vamorolone Form V is characterized by an X-ray powder diffraction pattern substantially, as shown in FIG. 8.

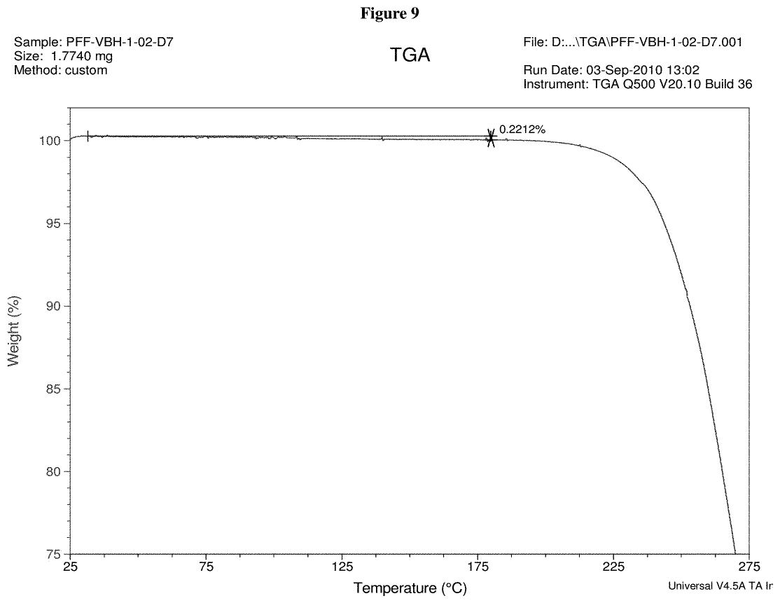

In some embodiments, vamorolone Form V is characterized by a thermogravimetric analysis profile showing less than about 0.5% weight loss below about 200° C. In some embodiments, vamorolone Form V is characterized by a thermogravimetric analysis profile substantially, as shown in FIG. 9.

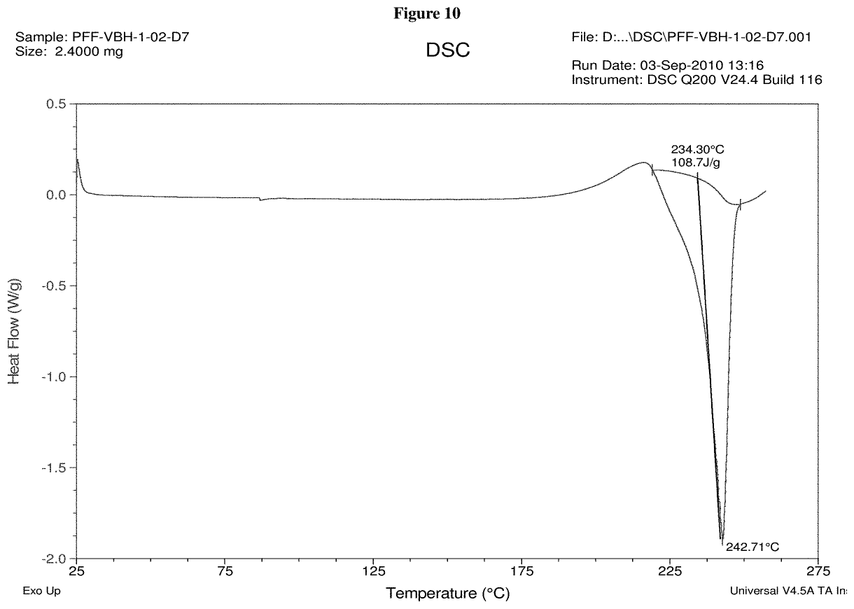

In some embodiments, vamorolone Form V is characterized by a melting event with an onset and peak temperatures of 234.3° C. and 242.7° C., respectively, as measured by differential scanning calorimetry. In some embodiments, vamorolone Form V is characterized by a differential scanning calorimetry trace substantially, as shown in FIG. 10.

In some embodiments, vamorolone Form V is prepared by a process comprising the step of slowly concentrating by evaporation at room temperature a solution of vamorolone in a solvent and isolating the resulting crystals. In some embodiments, the solvent is a mixture of ethyl acetate/dichloromethane.

### Vamorolone Form VI

Provided is a compound which is vamorolone Form VI.

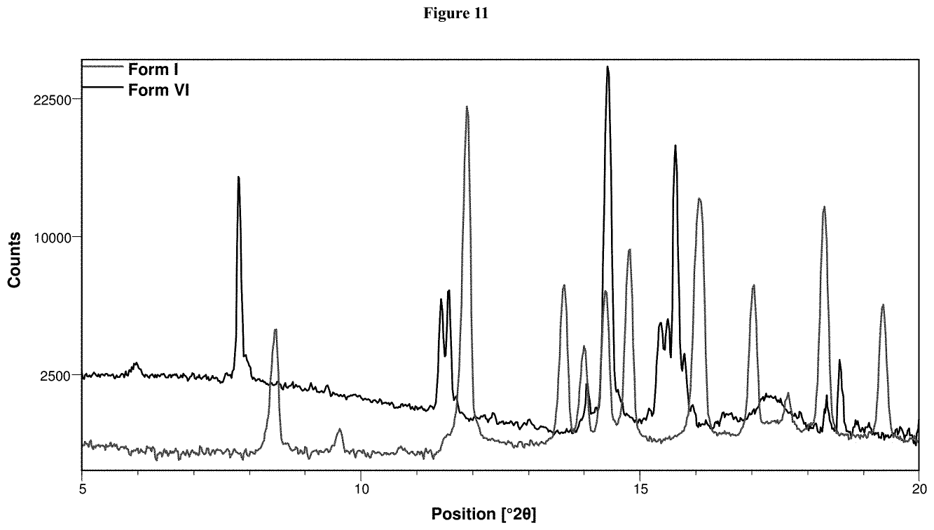

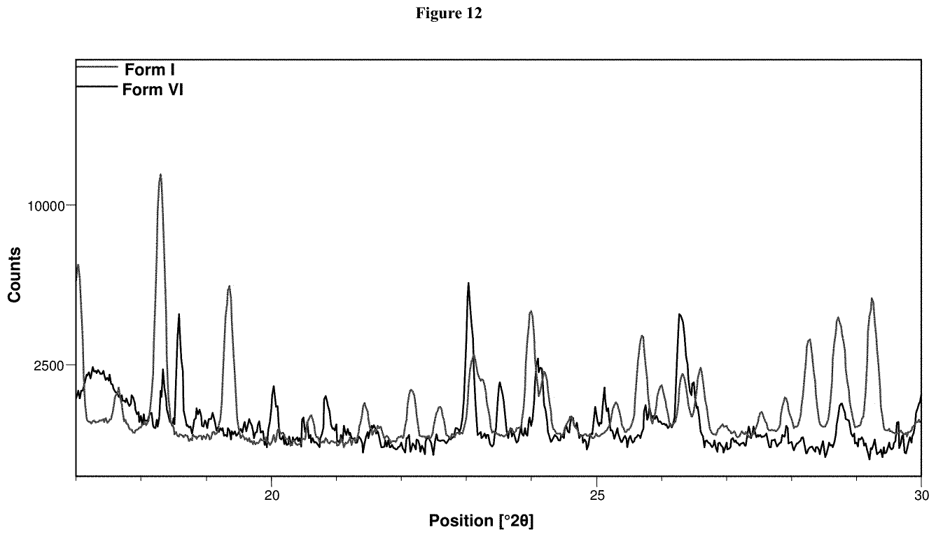

In some embodiments, vamorolone Form VI is characterized by an X-ray powder diffraction pattern comprising peaks, in terms of ° 2θ, at about 7.7, about 11.3, and about 11.5, with radiation Cu Kα. In some embodiments, vamorolone Form VI is characterized by an X-ray powder diffraction pattern comprising peaks, in terms of ° 2θ, at about 7.7, about 11.3, about 11.5, about 14.3, about 15.6 with radiation Cu Kα. In some embodiments, vamorolone Form VI is characterized by an X-ray powder diffraction pattern substantially, as shown in FIG. 11 or 12.

In some embodiments, vamorolone Form VI is prepared by a process comprising the step of slowly concentrating by evaporation at room temperature a solution of vamorolone in a solvent and isolating the resulting crystals. In some embodiments, the solvent is a mixture of acetonitrile/ethyl acetate.

### Vamorolone Form VII

Provided is a compound which is vamorolone Form VII.

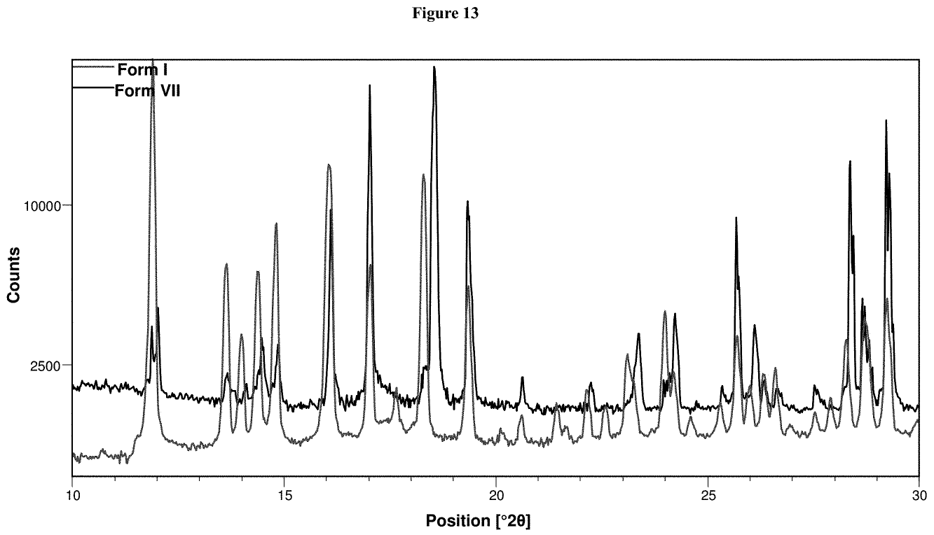

In some embodiments, vamorolone Form VII is characterized by an X-ray powder diffraction pattern comprising peaks, in terms of ° 2θ, at about 18.4, about 28.2, about 29.1, and about 29.3 with radiation Cu Kα. In some embodiments, vamorolone Form VII is characterized by an X-ray powder diffraction pattern comprising peaks, in terms of ° 2θ, at about 16.1, about 16.9, about 18.4, about 19.2, about 28.2, about 29.1, and about 29.3 with radiation Cu Kα. In some embodiments, vamorolone Form VII is characterized by an X-ray powder diffraction pattern substantially, as shown in FIG. 13.

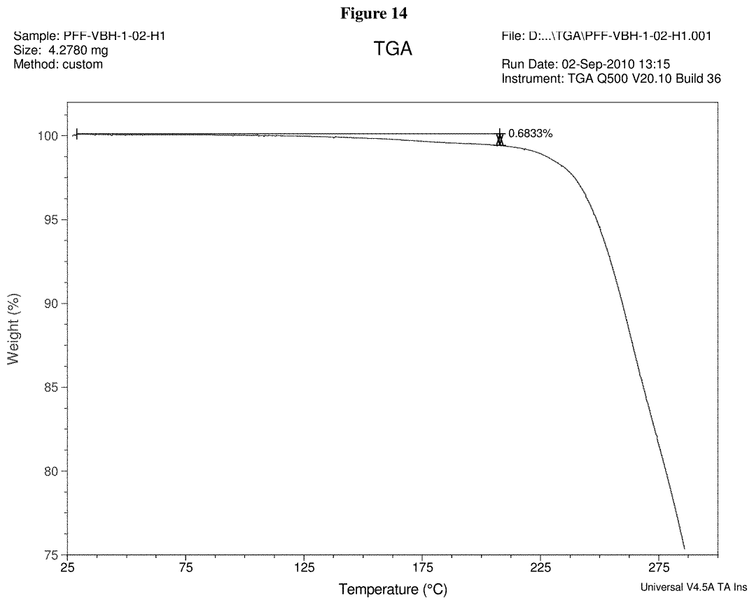

In some embodiments, vamorolone Form VII is characterized by a thermogravimetric analysis profile showing less than about 1% weight loss below about 225° C. In some embodiments, vamorolone Form VII is characterized by a thermogravimetric analysis profile substantially, as shown in FIG. 14.

In some embodiments, vamorolone Form VII is characterized by a melting event with an onset and peak temperatures of 227.0° C. and 236.9° C., respectively, as measured by differential scanning calorimetry.

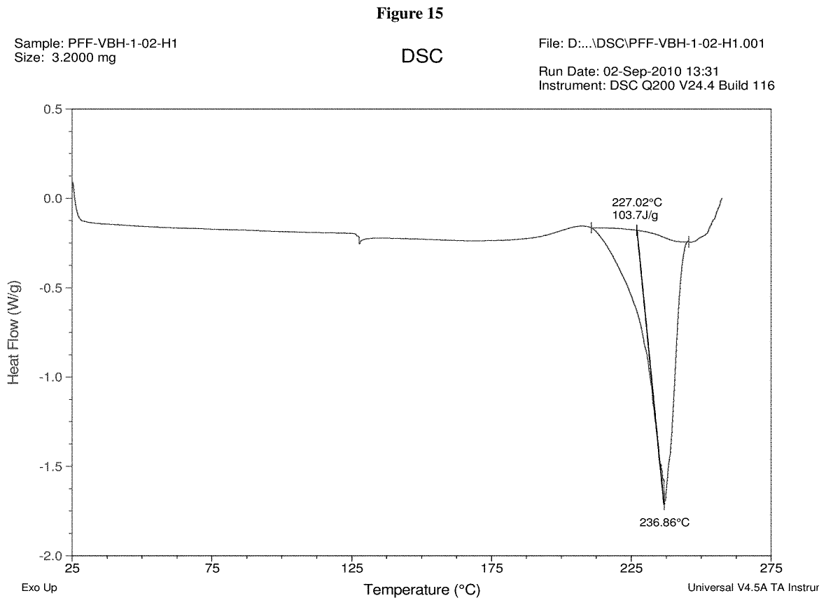

In some embodiments, vamorolone Form VII is characterized by a differential scanning calorimetry trace substantially, as shown in FIG. 15.

In some embodiments, vamorolone Form VII is prepared by a process comprising the step of slowly concentrating by evaporation at room temperature a solution of vamorolone in a solvent and isolating the resulting crystals. In some embodiments, the solvent is a mixture of acetonitrile/2-butanone, acetonitrile/dichloromethane, or dichloromethane/nitromethane.

### Vamorolone Form VIII

Provided is a compound which is vamorolone Form VIII.

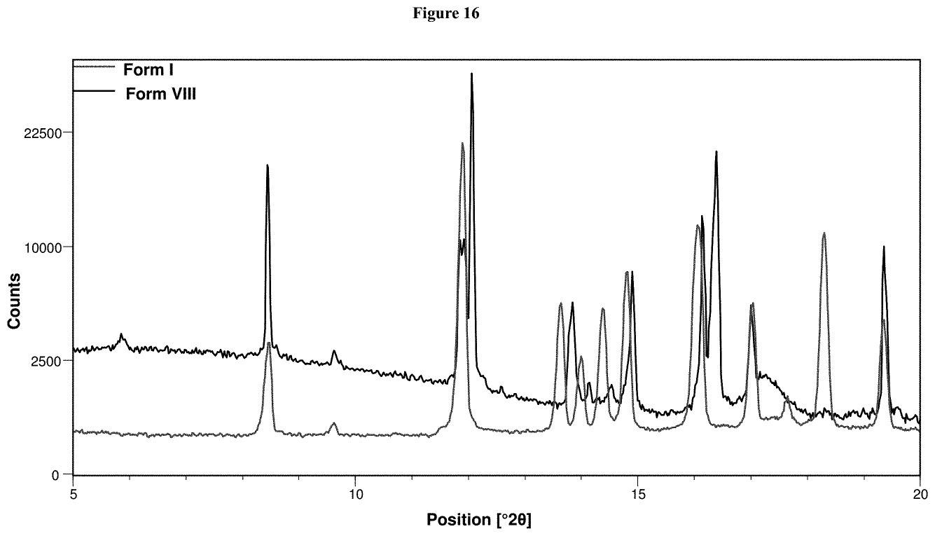

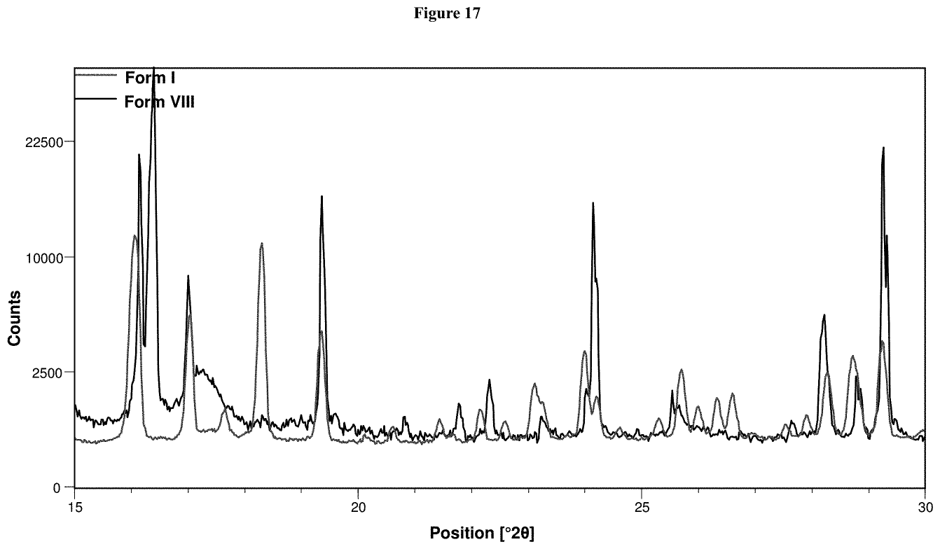

In some embodiments, vamorolone Form VIII is characterized by an X-ray powder diffraction pattern comprising peaks, in terms of ° 2θ, at about 16.1 and about 16.3, with radiation Cu Kα. In some embodiments, vamorolone Form VIII is characterized by an X-ray powder diffraction pattern comprising peaks, in terms of ° 2θ, at about 8.4, about 12.0, about 16.1, about 16.3, and about 29.2 with radiation Cu Kα. In some embodiments, vamorolone Form VIII is characterized by an X-ray powder diffraction pattern substantially, as shown in FIG. 16 or 17.

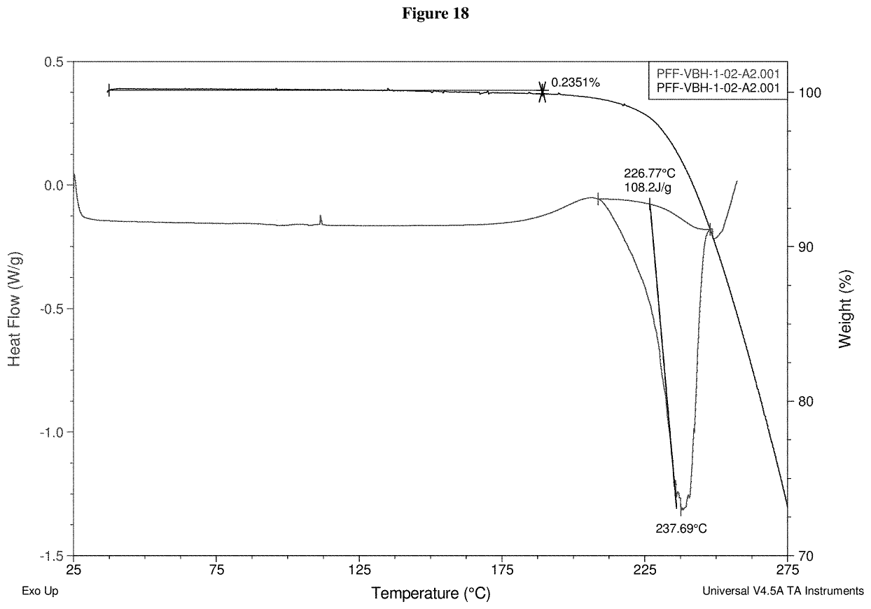

In some embodiments, vamorolone Form VIII is characterized by a thermogravimetric analysis profile showing less than about 0.5% weight loss below about 200° C. In some embodiments, vamorolone Form VIII is characterized by a thermogravimetric analysis profile substantially, as shown in FIG. 18.

In some embodiments, vamorolone Form VIII is characterized by a melting event with an onset and peak temperatures of 226.8° C. and 237.7° C., respectively, as measured by differential scanning calorimetry. In some embodiments, vamorolone Form VIII is characterized by a differential scanning calorimetry trace substantially, as shown in FIG. 18.

In some embodiments, vamorolone Form VIII is prepared by a process comprising the step of slowly concentrating by evaporation at room temperature a solution of vamorolone in a solvent and isolating the resulting crystals. In some embodiments, the solvent is a mixture of acetonitrile/acetone, acetone/dichloromethane, ethyl acetate/tetrahydrofuran, methanol/2-propanol, or tetrahydrofuran/toluene.

### Pharmaceutical Compositions

While vamorolone can be administered as a raw chemical, it is also possible to present it as a pharmaceutical formulation. Accordingly, provided herein are pharmaceutical compositions comprising at least one polymorphic form of vamorolone and at least one pharmaceutically acceptable excipient.

In some embodiments, the polymorphic form is vamorolone Form II. In some embodiments, the polymorphic form is vamorolone Form III. In some embodiments, the polymorphic form is vamorolone Form IV. In some embodiments, the polymorphic form is vamorolone Form V. In some embodiments, the polymorphic form is vamorolone Form VI. In some embodiments, the polymorphic form is vamorolone Form VII. In some embodiments, the polymorphic form is vamorolone Form VIII. In some embodiments, the polymorphic form is a mixture comprising at least two polymorphic forms of vamorolone chosen from Form II, Form III, Form IV, Form V, Form VI, Form VII, and Form VIII.

The excipient(s) or carrier(s) should be “acceptable” in the sense of being compatible with the other ingredients of the formulation and not deleterious to the recipient thereof. Proper formulation depends on the route of administration chosen. Any well-known techniques, carriers, and excipients may be used as suitable and as understood in the art, e.g., in Remington's Pharmaceutical Sciences. The pharmaceutical compositions disclosed herein may be manufactured in any manner known in the art, e.g., through conventional mixing, dissolving, granulating, dragee-making, levigating, emulsifying, encapsulating, entrapping, or compression processes.

The formulations include those suitable for oral, parenteral (including subcutaneous, intradermal, intramuscular, intravenous, intraarticular, and intramedullary), intraperitoneal, transmucosal, transdermal, rectal, and topical (including dermal, buccal, sublingual, and intraocular) administration, although the most suitable route may depend upon for example the condition and disorder of the recipient. The formulations may conveniently be presented in unit dosage form and may be prepared by any of the methods well known in the art of pharmacy. Typically, these methods include bringing into association a compound of the subject disclosure or a pharmaceutically acceptable salt, ester, amide, prodrug, or solvate thereof (“active ingredient”) with the carrier that constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of vamorolone suitable for oral administration may be presented as discrete units such as capsules, cachets, or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary, or paste.

Pharmaceutical preparations that can be used orally include tablets, push-fit capsules made of gelatin, as well as soft, sealed capsules made of gelatin, and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface-active, or dispersing agents. Molded tablets may be made by molding in a suitable machine a mixture of the powdered compound moistened with an inert liquid diluent. The tablets may optionally be coated or scored and may be formulated to provide a slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings to identify or characterize different combinations of active compound doses.

The compounds may be formulated for parenteral administration by injection, e.g., by bolus injection or continuous infusion. Formulations for injection may be presented in unit dosage form, e.g., in ampoules or multi-dose containers, with an added preservative. The compositions may take such forms as suspensions, solutions, or emulsions in oily or aqueous vehicles and may contain formulatory agents such as suspending, stabilizing, and/or dispersing agents. The formulations may be presented in unit-dose or multi-dose containers, for example, sealed ampoules and vials. They may be stored in powder form or in a freeze-dried (lyophilized) condition requiring only the addition of the sterile liquid carrier, for example, saline or sterile pyrogen-free water, immediately before use. Extemporaneous injection solutions and suspensions may be prepared from sterile powders, granules, and tablets of the kind previously described.

Formulations for parenteral administration include aqueous and non-aqueous (oily) sterile injection solutions of the active compounds, which may contain antioxidants, buffers, bacteriostats, and solutes which render the formulation isotonic with the blood of the intended recipient; and aqueous and non-aqueous sterile suspensions, which may include suspending agents and thickening agents. Suitable lipophilic solvents or vehicles include fatty oils such as sesame oil or synthetic fatty acid esters, such as ethyl oleate or triglycerides, or liposomes. Aqueous injection suspensions may contain substances that increase the suspension's viscosity, such as sodium carboxymethyl cellulose, sorbitol, or dextran. Optionally, the suspension may also contain suitable stabilizers or agents that increase the compounds' solubility to prepare highly concentrated solutions.

For oral or parenteral use, the compounds may be formulated as nanoparticle preparations. Such nanoparticle preparations can include, for example, nanosphere encapsulations of active compounds, inactive nanoparticles to which active compounds can be tethered, or nanoscale powders of active compounds. Nanoparticle preparations can be used to increase the bioavailability of the active compounds, control the rate of release of the active compounds, or deliver active compounds to a location in the body. See A. Dove, “An Easy Pill to Swallow,” Drug Discovery & Development Magazine: 11(11), November 2008, pp. 22-24.

In addition to the formulations described previously, the compounds may also be formulated as a depot preparation. Such long-acting formulations may be administered by implantation (for example, subcutaneously or intramuscularly) or intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated conventionally. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Vamorolone may be administered topically, that is, by non-systemic administration. This includes applying vamorolone externally to the epidermis or the buccal cavity and installing such a compound into the ear, eye, and nose. The compound does not significantly enter the bloodstream. In contrast, systemic administration refers to oral, intravenous, intraperitoneal, and intramuscular administration.

Formulations suitable for topical administration include liquid or semi-liquid preparations suitable for penetration through the skin to the site of inflammation, such as gels, liniments, lotions, creams, ointments or pastes, and drops suitable for administration to the eye, ear, or nose. The active ingredient for topical administration may comprise, for example, from 0.001% to 10% w/w (by weight) of the formulation. In some embodiments, the active ingredient may comprise as much as 10% w/w. In other embodiments, it may comprise less than 5% w/w. In some embodiments, the active ingredient may comprise from 2% w/w to 5% w/w. In other embodiments, it may comprise from 0.1% to 1% w/w of the formulation.

Formulations for topical administration in the mouth, for example, buccally or sublingually, include lozenges comprising the active ingredient in a flavored basis such as sucrose and acacia or tragacanth and pastilles comprising the active ingredient in a basis such as gelatin and glycerin or sucrose and acacia.

For administration by inhalation, compounds may be conveniently delivered from an insufflator, nebulizer pressurized packs, or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, the compounds, according to the disclosure, may take the form of a dry powder composition, for example, a powder mix of the compound and a suitable powder base such as lactose or starch. The powder composition may be presented in unit dosage form, such as capsules, cartridges, gelatin, or blister packs from which the powder may be administered with the aid of an inhalator or insufflator.

Unit dosage formulations contain an effective dose, as herein below recited, or an appropriate fraction thereof, of the active ingredient.

In addition to the ingredients mentioned above, the formulations described above may include other agents conventional in the art regarding the type of formulation in question. For example, those suitable for oral administration may include flavoring agents.

Compounds may be administered orally or via injection at a dose of from 0.1 to 500 mg/kg per day. The dose range for adult humans is generally from 5 mg to 2 g/day. In some embodiments, the dose range is 0.25 to 6.0 mg/kg/day, such as 0.25 mg/kg/day, 0.75 mg/kg/day, 2.0 mg/kg/day, or 6.0 mg/kg/day. Tablets or other forms of presentation provided in discrete units may conveniently contain one or more compounds that are effective at such dosage or as a multiple of the same, for instance, units containing 5 mg to 500 mg, usually around 10 to 200 mg.

The amount of active ingredient combined with the carrier materials to produce a single dosage form will vary depending upon the host treated and the particular mode of administration.

The compounds can be administered in various modes, e.g., orally, topically, or by injection. The precise amount of compound administered to a patient will be the responsibility of the attendant physician. The specific dose level for any particular patient will depend upon a variety of factors, including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one compound described herein (or a pharmaceutically acceptable salt, ester, or prodrug thereof) combined with another therapeutic agent. By way of example only, if one side effect experienced by a patient upon receiving one compound herein is hypertension, then it may be appropriate to administer an anti-hypertensive agent in combination with the initial therapeutic agent. Or, by way of example only, the therapeutic effectiveness of one compound described herein may be enhanced by administration of an adjuvant (i.e., by itself, the adjuvant may only have minimal therapeutic benefit, but in combination with another therapeutic agent, the overall therapeutic benefit to the patient is enhanced). Or, by way of example only, the benefit experienced by a patient may be increased by administering one compound described herein with another therapeutic agent (which also includes a therapeutic regimen) that also has therapeutic benefit. By way of example only, in a treatment for diabetes involving administration of one compound described herein, increased therapeutic benefit may result by also providing the patient with another therapeutic agent for diabetes. In any case, regardless of the disease, disorder, or condition being treated, the overall benefit experienced by the patient may simply be additive of the two therapeutic agents, or the patient may experience a synergistic benefit.

In any case, the multiple therapeutic agents (at least one of which is vamorolone) may be administered in any order or even simultaneously. If simultaneous, the multiple therapeutic agents may be provided in a single, unified form or multiple forms (by example only, either as a single pill or two separate pills). One therapeutic agent may be given in multiple doses, or both may be given multiple doses. If not simultaneous, the timing between the multiple doses may be any duration of time ranging from a few minutes to four weeks.

### Methods of Use

Also provided are methods for treating a disease having a significant and pathologic inflammatory component that can be addressed by inhibition of NF-κB in a human or animal subject in need of such treatment comprising administering to the subject an amount of a polymorphic form of vamorolone effective to reduce or prevent the disease in the subject, optionally in combination with at least one additional agent for the treating the disease that is known in the art.

Specific diseases to be treated by the compounds, compositions, and methods disclosed herein include acid-induced lung injury, acne (PAPA), acute respiratory distress syndrome, ageing, AIDS, HIV-1, alcoholic hepatitis, alcoholic liver disease, allergic bronchopulmonary aspergillosis, Alzheimer's disease, amyotropic lateral sclerosis, angina pectoris, anhidrotic ecodermal dysplasia-ID, ankylosing spondylitis, antiphospholipid syndrome, aphthous stomatitis, appendicitis, arthritis, asthma, allergen induced asthma, non-allergen induced asthma, atherosclerosis, atopic dermatitis, autoimmune diseases, Behcet's disease, Bell's palsy, Blau syndrome, bronchiolitis, cancer, cardiac hypertrophy, catabolic disorders, cataracts, cerebral aneurysm, chronic heart failure, chronic lung disease (including of prematurity), chronic obstructive pulmonary disease, colitis, ulcerative colitis, complex regional pain syndrome, connective tissue diseases, crohn's disease, cryopyrin-associated periodic syndromes, cyrptococcosis, cystic fibrosis, deficiency of the interleukin-1-receptor antagonist (DIRA), dermatitis, dermatomyositis, endometriosis, endotoxemia, familial amyloidotic polyneuropathy, familial cold urticaria, familial mediterranean fever, fetal growth retardation, glaucoma, glomerular disease, glomerular nephritis, gut diseases, head injury, headache, hearing loss, heart disease, Henoch-Scholein purpura, hepatitis, hereditary periodic fever syndrome, herpes zoster and simplex, Huntington's disease, hyaline membrane disease, hypercholesterolemia, hyperimmunoglobulinemia D with recurrent fever (HIDS), hypoplastic and other anemias, incontinentia pigmenti, infectious mononucleosis, inflammatory bowel disease, inflammatory lung disease, inflammatory neuropathy, inflammatory pain, irritant-induced inflammation, plant irritant-induced inflammation, poison ivy/urushiol oil-induced inflammation, chemical irritant-induced inflammation, bee sting-induced inflammation, insect bite-induced inflammation, ischemia/reperfusion, kidney disease, kidney injury caused by parasitic infections, leptospiriosis, leukemia, lung injury, lupus, lupus nephritis, lymphoma, meningitis, mesothelioma, Muckle-Wells syndrome (urticaria deafness amyloidosis), multiple sclerosis, muscle wasting, muscular dystrophy, mycosis fungoides, myelodysplastic syndrome, myocarditis, myositis, nasal sinusitis, necrotizing enterocolitis, neonatal onset multisystem inflammatory disease (NOMID), nephrotic syndrome, neuritis, neuropathological diseases, obesity, ocular allergy, osteoarthritis, otitis media, Paget's disease, pain, pancreatitis, Parkinson's disease, pericarditis, periodic fever, periodonitis, peritoneal endometriosis, pertussis, pharyngitis and adenitis (PFAPA syndrome), pneumocystis infection, polyarteritis nodosa, polycystic kidney disease, polymyositis, psoriasis, psychosocial stress diseases, pulmonary disease, pulmonary fibrosis, pulmonary hypertension, pyoderma gangrenosum, pyogenic sterile arthritis, renal disease, retinal disease, rheumatic disease, rheumatoid arthritis, sarcoidosis, seborrhea, sepsis, silica-induced diseases, Sjogren's syndrome, skin diseases, sleep apnea, solid tumors, spinal cord injury, stroke, subarachnoid hemorrhage, sunburn, burns, thrombocytopenia, TNF receptor associated periodic syndrome (TRAPS), toxoplasmosis transplant, organ transplant, tissue transplant, traumatic brain injury, tuberculosis, type 1 diabetes, type 2 diabetes, uveitis.

In some embodiments, the disease is chosen from acute lymphocytic leukemia, Addison's disease, adrenal hyperplasia, adrenocortical insufficiency, allergic conjunctivitis, alopecia, amyloidosis, angioedema, anterior segment inflammation, autoimmune hepatitis, Behcet's syndrome, berylliosis, bone pain, bursitis, carpal tunnel syndrome, chorioretinitis, chronic lymphocytic leukemia, corneal ulcer, diffuse intrinsic pontine glioma, epicondylitis, erythroblastopenia, gout, gouty arthritis, graft-versus-host disease, hemolytic anemia, Hodgkin's disease, hypercalcemia, hyperammonemia, hypoplastic anemia, idiopathic thrombocytopenic purpura, iritis, juvenile rheumatoid arthritis, keratitis, kidney transplant rejection prophylaxis, Loeffler's syndrome, mixed connective tissue disease, myasthenia gravis, mycosis fungoides, optic neuritis, pemphigus, pneumonia, pneumonitis, polychondritis, psoriasis, rheumatic carditis, severe pain, sickle cell, sickle cell anemia, Stevens-Johnson syndrome, temporal arteritis, tenosynovitis, thyroiditis, urticarial, Wegener's granulomatosis, and weight loss.

In some embodiments, the disease is asthma or chronic obstructive pulmonary disease

In some embodiments, the disease is Sjogren's syndrome.

In some embodiments, the disease is arthritis.

In some embodiments, the disease is muscular wasting.

In some embodiments, the muscular wasting disease is muscular dystrophy.

In some embodiments, the muscular dystrophy is chosen from Duchenne muscular dystrophy, Becker muscular dystrophy, limb-girdle muscular dystrophy, congenital muscular dystrophy, facioscapulohumeral muscular dystrophy, myotonic muscular dystrophy, oculopharyngeal muscular dystrophy, distal muscular dystrophy, and Emery-Dreifuss muscular dystrophy.

In some embodiments, the muscular dystrophy is Duchenne muscular dystrophy.

Besides being useful for human treatment, certain compounds and formulations disclosed herein may also be useful for veterinary treating companion animals, exotic animals, and farm animals, including mammals, rodents, and the like. More animals include horses, dogs, and cats.

All references, patents, or applications, U.S. or foreign, cited in the application are hereby incorporated by reference as if written herein in their entireties. Where any inconsistencies arise, material literally disclosed herein controls.

Further embodiments include the embodiments disclosed in the following Examples, which are not to be construed as limiting in any way.

### EXAMPLES

The following examples are included to demonstrate some embodiments of the disclosure. It should be appreciated by those of skill in the art that the techniques disclosed in the examples represent techniques discovered by the inventors to function well in the practice of the disclosure. Those of skill in the art should, however, in light of the present disclosure, appreciate that many changes can be made in the specific embodiments disclosed and still obtain a like or similar result without departing from the spirit and scope of the disclosure; therefore all matter set forth is to be interpreted as illustrative and not in a limiting sense.

### Example 1: Polymorphic Screen

### Experimental Procedures

The solid-state form screening (SSFS) of crystalline drug substance vamorolone was performed using an array of 13 solvents, Table 1, using slow evaporation for crystallization conditions, together with a binary mixture (Table 2).

TABLE 1

| # | Solvent |
|---|---|
| 1 | Acetonitrile |
| 2 | Acetone |
| 3 | ethanol |
| 4 | Ethyl acetate |
| 5 | Methanol |
| 6 | 2-propanol |
| 7 | THF |
| 8 | Toluene |
| 9 | 2-Butanone |
| 10 | dichloromethane |
| 11 | Heptane |
| 12 | Water |
| 13 | Nitromethane |

### Preparation of Drug Solutions

Drug substance was weighted into glass vials. The vials were then each filled with about 5 mL of the desired solvents, stirred (vortex mixer), and heated to 45° C. Residue suspensions consisting of primarily solid particulates were later used for slurry studies.

### Filtration of Drug Solutions/Suspensions

All drug solutions/suspensions were manually filtered into clean glass vials using plastic non-contaminating syringes equipped with 0.22-μm nylon filter cartridges. The filtrates were then used for crystallization/precipitation studies, as outlined below.

### Well Plate Preparation/Solvent Distribution/Crystallization

The saturated drug solutions (filtrates) were distributed in a 96-well plate, according to the solvent matrix in Table 2. The solvents in the plate were allowed to evaporate in an operating laboratory fume hood under ambient temperature and humidity conditions. The plate was covered for the slow solvent evaporation (crystallization) condition. During the process of crystallization, the plate was visually examined, and any solid material was analyzed by an imaging system, powder x-ray diffraction (PXRD), differential scanning calorimetry (DSC), and thermogravimetric analysis (TGA), as deemed appropriate based on the amount of sample.

### Slurry Studies

Residue suspensions were allowed to dry in glass vials in an operating laboratory fume hood under ambient temperature and humidity conditions. These solids were analyzed by powder x-ray diffraction (PXRD), differential scanning calorimetry (DSC), and thermogravimetric analysis (TGA), as deemed appropriate based on the amount of sample.

### Excess Filtrate Crystallizations/Precipitations

After 96-well plate preparation, residual filtrates were allowed to evaporate to dryness in glass test tubes in an operating laboratory fume hood under ambient conditions of temperature and humidity. Any solids will be analyzed by powder x-ray diffraction (PXRD), differential scanning calorimetry (DSC), and thermogravimetric analysis (TGA), as deemed appropriate based on the amount of sample.

Table 2 lists the solvent distribution matrix for both well plates.

TABLE 2

| Well | ID | Solvent 1 | Solvent 2 |
|---|---|---|---|
| A1 | 1 | Acetonitrile | Acetonitrile |
| A2 | 14 | Acetonitrile | Acetone |
| A3 | | Acetonitrile | ethanol |
| A4 | 15 | Acetonitrile | Ethyl acetate |
| A5 | 16 | Acetonitrile | Methanol |
| A6 | | Acetonitrile | 2-propanol |
| A7 | 17 | Acetonitrile | THF |
| A8 | 18 | Acetonitrile | Toluene |
| A9 | 19 | Acetonitrile | 2-Butanone |
| A10 | 20 | Acetonitrile | dichloromethane |
| A11 | 21 | Acetonitrile | Heptane |
| A12 | 22 | Acetonitrile | Water |
| B1 | 23 | Acetonitrile | Nitromethane |
| B2 | 2 | Acetone | Acetone |
| B3 | | Acetone | ethanol |
| B4 | 24 | Acetone | Ethyl acetate |
| B5 | 25 | Acetone | Methanol |
| B6 | | Acetone | 2-propanol |
| B7 | 26 | Acetone | THF |
| B8 | 27 | Acetone | Toluene |
| B9 | 28 | Acetone | 2-Butanone |
| B10 | 29 | Acetone | dichloromethane |
| B11 | 30 | Acetone | Heptane |
| B12 | 31 | Acetone | Water |
| C1 | 32 | Acetone | Nitromethane |
| C2 | 3 | ethanol | ethanol |
| C3 | | ethanol | Ethyl acetate |
| C4 | 40 | ethanol | Methanol |
| C5 | | ethanol | 2-propanol |
| C6 | | ethanol | THF |
| C7 | | ethanol | Toluene |
| C8 | | ethanol | 2-Butanone |
| C9 | | ethanol | dichloromethane |
| C10 | | ethanol | Heptane |
| C11 | | ethanol | Water |
| C12 | | ethanol | Nitromethane |
| D1 | 4 | Ethyl acetate | Ethyl acetate |
| D2 | 33 | Ethyl acetate | Methanol |
| D3 | | Ethyl acetate | 2-propanol |
| D4 | 34 | Ethyl acetate | THF |
| D5 | 35 | Ethyl acetate | Toluene |
| D6 | 36 | Ethyl acetate | 2-Butanone |
| D7 | 37 | Ethyl acetate | dichloromethane |
| D8 | 38 | Ethyl acetate | Heptane |
| D9 | | Ethyl acetate | Water |
| D10 | 39 | Ethyl acetate | Nitromethane |
| D11 | 5 | Methanol | Methanol |
| D12 | 41 | Methanol | 2-propanol |
| E1 | 42 | Methanol | THF |
| E2 | 43 | Methanol | Toluene |
| E3 | 44 | Methanol | 2-Butanone |
| E4 | 45 | Methanol | dichloromethane |
| E5 | | Methanol | Heptane |
| E6 | 46 | Methanol | Water |
| E7 | 47 | Methanol | Nitromethane |
| E8 | 6 | 2-propanol | 2-propanol |
| E9 | | 2-propanol | THF |
| E10 | | 2-propanol | Toluene |
| E11 | | 2-propanol | 2-Butanone |
| E12 | | 2-propanol | dichloromethane |
| F1 | | 2-propanol | Heptane |
| F2 | | 2-propanol | Water |
| F3 | | 2-propanol | Nitromethane |
| F4 | 7 | THF | THF |
| F5 | 48 | THF | Toluene |
| F6 | 49 | THF | 2-Butanone |
| F7 | 50 | THF | dichloromethane |
| F8 | 51 | THF | Heptane |
| F9 | 52 | THF | Water |
| F10 | 53 | THF | Nitromethane |
| F11 | 8 | Toluene | Toluene |
| F12 | 54 | Toluene | 2-Butanone |
| G1 | 55 | Toluene | dichloromethane |
| G2 | 56 | Toluene | Heptane |
| G3 | | Toluene | Water |
| G4 | 57 | Toluene | Nitromethane |
| G5 | 9 | 2-Butanone | 2-Butanone |
| G6 | 58 | 2-Butanone | dichloromethane |
| G7 | 59 | 2-Butanone | Heptane |
| G8 | | 2-Butanone | Water |
| G9 | 60 | 2-Butanone | Nitromethane |
| G10 | 10 | dichloromethane | dichloromethane |
| G11 | 61 | dichloromethane | Heptane |
| G12 | | dichloromethane | Water |
| H1 | 62 | dichloromethane | Nitromethane |
| H2 | 11 | Heptane | Heptane |
| H3 | | Heptane | Water |
| H4 | 63 | Heptane | Nitromethane |
| H5 | 12 | Water | Water |
| H6 | | Water | Nitromethane |
| H7 | 13 | Nitromethane | Nitromethane |

### Thermal Treatment

### Cold Crystallization

The melting of vamorolone was performed in a DSC apparatus at a heating rate of 10° C./min. The DSC furnace was programmed to cool at 10° C./min down to −40° C. A second heating cycle at a heating rate of 10° C./min was performed.

### Sample Analysis

Solid samples were analyzed using various analytical techniques by Pharmaron personnel, Beijing, China using Polarizing microscope (PLM), X-ray diffraction (pXRD), hot-stage X-ray diffraction, differential scanning calorimetry (DSC), thermogravimetry (TGA), NMR, and infrared spectrometry.

### Polarizing Microscope Analysis (PLM)

PLM analyses were conducted on a Nikon Instruments Eclipse 80i. The images were captured by a DS camera and transmitted to the computer. The photos were processed using a NIS-Elements D3.0 software.

Powder X-ray Diffraction (pXRD)

The solid samples were examined using an X-ray diffractometer (Bruker D8 advance). The system is equipped with highly-parallel x-ray beams (Göbel Mirror) and a LynxEye detector. The samples were scanned from 3 to 40° 20 at a step size of 0.02° 20 and a time per step of 19.70 seconds. The tube voltage and current were 45 kV and 40 mA, respectively. The sample was transferred from the sample container onto a zero background XRD-holder and gently ground.

### TGA Analysis

TGA analyses were carried out on a TA Instruments TGA Q500. Approximately 2.0 mg of samples were placed in a tared platinum or aluminum pan, automatically weighed, and inserted into the TGA furnace. The samples were heated at 10° C./min to a final temperature of 300° C. The purge gas is nitrogen for balance at 40 mL/min and the sample at 60 mL/min, respectively.

### DSC Analysis

DSC analyses were conducted on a TA Instruments Q200. The calibration standard was indium. A sample of 2.0 mg in weight was placed into a TA DSC pan, and weight was accurately recorded. Crimped pans were used for analysis, and the samples were heated under nitrogen (50 mL/min) at a rate of 10° C./min, up to a final temperature of 260° C.

### Infrared Spectrometry (FTIR)

FTIR analyses were conducted on a Thermo Instruments IS10. Mix the compound with potassium bromide and press to a pellet (Ph. Eur.). Record the FTIR spectrum of the pellet between 4000 cm⁻¹ and 400 cm⁻¹ using 32 scans at a 1 cm⁻¹ resolution.

### NMR Analysis

Proton NMR was used to characterize degrading the compound upon heating in DSC cells. Proton NMR was performed using Bruker Advance 300 equipped with an automated sample (B-ACS 120). DMSO-d₆ was used as a solvent for NMR analysis.

### Results and Discussion

A total of eight polymorphs were identified during this study (seen in Table 3).

TABLE 3

| Form | Sample ID |
|---|---|
| I | A1, A5, A7, A11, A12, B1, B4, B5, B7, B8, B9, B11, B12, |
| | C1, C2, C4, D5, D8, D10, D11, D12, E1, E2, E3, E4, E6, E7, |
| | F4, F6, F8, F10, F12, G1, G4, G5, G4, G7, G11, H4, H7 |
| II | DSC 206 and hot-stage pXRD |
| III | G6, G10 |
| IV | B2, D2 |
| V | D7 |
| VI | A4 |
| VII | A9, A10, H1 |
| VIII | A2, B10, D4, D12,, F5, |

Form II was observed in DSC thermal treatment; however, the pXRD pattern (FIGS. 1 and 2) was only obtained using hot-stage pXRD. Even at 205° C., the pXRD pattern is almost as same as the form I below 15° 20, while a more pronounced difference showed above 15° 20.

Form III is obtained from solvent crystallization from either dichloromethane or 2-butanone/dichloromethane (see Table 3). The pXRD pattern of Form III is shown in FIGS. 3 and 4.

Form IV is generated from solvent crystallization from either acetone or ethyl acetate/methanol (Table 3), shown in FIGS. 5 and 6. TGA showed that there is little weight loss (<0.5%), as shown in FIG. 7.

Form V resulted from solvent crystallization from the mixture of ethyl acetate and dichloromethane (see Table 3). The pXRD pattern of Form V is shown in FIG. 8. The TGA and DSC for Form V are shown in FIGS. 9 and 10, respectively.

Form VI was obtained from solvent crystallization from the mixture of ethyl acetate and acetonitrile (Table 3). The pXRD pattern of Form VI is shown in FIGS. 11 and 12.

Form VII was obtained from solvent crystallization from several combinational solvents (Table 3). The pXRD pattern of Form VII is shown in FIG. 13. FIGS. 14 and 15 show the TGA and DSC profiles, respectively, of Form VII.

Form VIII was from solvent crystallization from numerous solvents (Table 3). The pXRD of Form VIII is shown in FIGS. 16 and 17. FIG. 18 shows the TGA and DSC profiles of Form VIII.

### Slurry Conversion Study

The slurry in water was performed to study the relationship of polymorphs.

A solid sample isolated from a DSC cell heated at 206° C. was mixed with Form I at a 1:1 ratio, which was then suspended in water 20 mg in 1 mL. The suspension was kept on shaking for 3 days. The solid was collected through filtration and was then characterized. The pXRD pattern after slurry in water is similar to Form I.

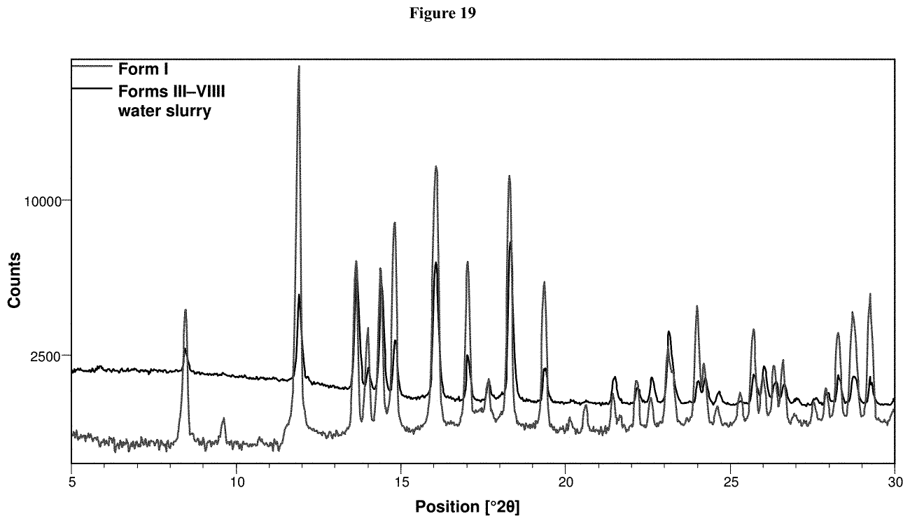

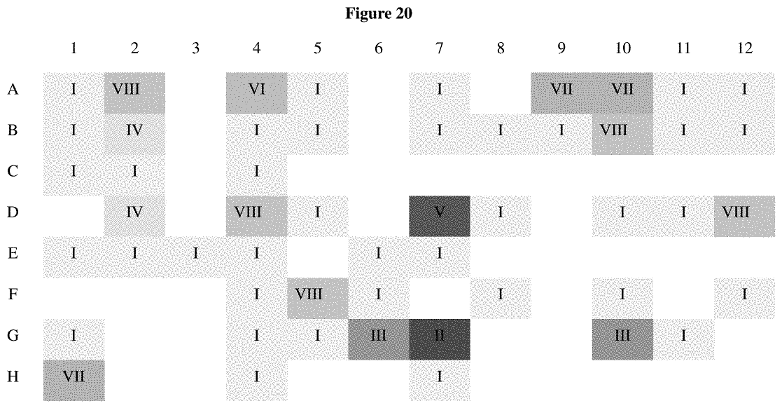

Form I was mixed with various amounts of Forms III, IV, V, VI, VII, and VIII, depending on the limitation of samples, then suspended in water. The slurry was shaken for 3 days. The solid was collected through filtration and characterized. The pXRD pattern of the mixture after the slurry is similar to the Form I's, as shown in FIG. 19.

Form II vs. Form I

Form II was observed in DSC thermal experiment. However, the pXRD pattern obtained after the sample was cooled down to room temperature is similar to the initial material. This result suggested that Form I and Form II are enantiotropically related; i.e., below the transition temperature, Form I is the most stable form while Form II is most stable above the transition temperature. It is estimated from DSC and hot-stage pXRD data that the transition temperature could well be above 100° C. Form I is described in U.S. patent application Ser. No. 16/811,973 (Attorney Docket No. VLD0007-201-US, which is incorporated herein by reference in its entirety for all purposes.)

### Conclusions

In total, eight polymorphs were identified during this study. Form II was only observed by DSC thermal treatment of Form I; however, the pXRD pattern of Form II was obtained using hot-stage pXRD. Forms III-VIII were obtained from solvent crystallization.

### Example 2

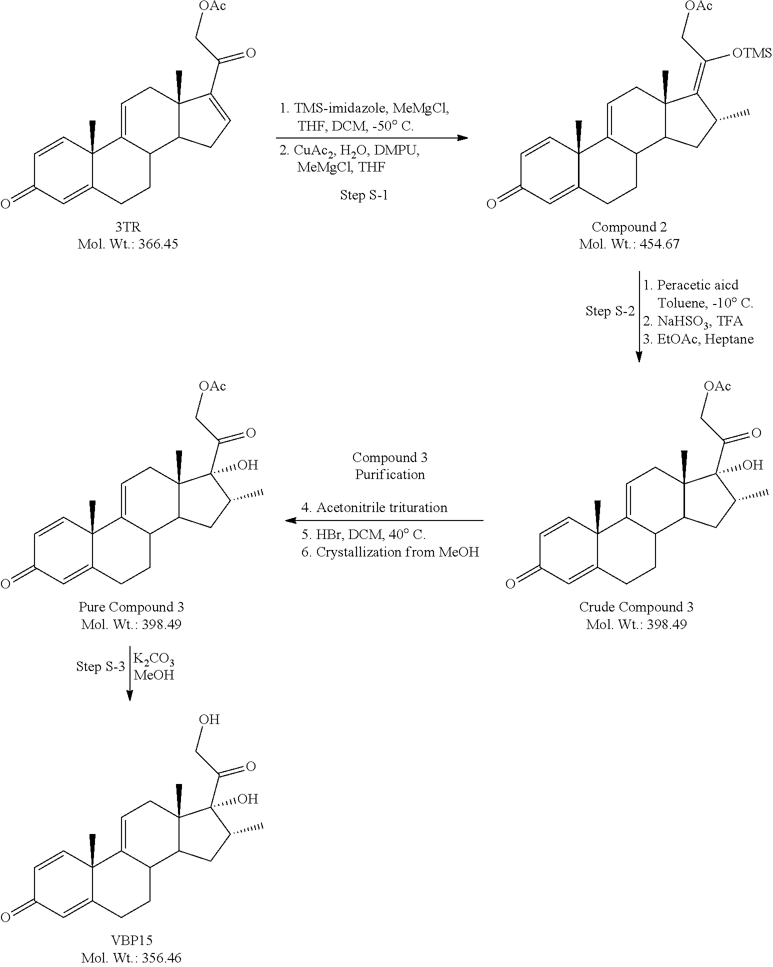

### Compound 2 Preparation

3-TR (100 g, 273 mmol), dichloromethane (DCM, 500 mL), and tetrahydrofuran (THF, 400 mL) were charged to a reaction flask under nitrogen. To this was charged trimethylsilyl imidazole (TMS-imidazole, 65.3 g, 466 mmol, 1.7 eq). The resulting mixture was stirred at room temperature for 3 hours.

In a separate flask, copper acetate monohydrate (5.4 g, 27 mmol), tetrahydrofuran (400 ml), and 1,3-dimethyl-3,4,5,6-tetrahydro-2(1H)-pyrimidinone (DMPU, 53.3 g, 416 mmol) were combined and stirred at room temperature for approximately 3 hours. The blue mixture was subsequently cooled to −50° C. and was added methyl magnesium chloride solution (27 ml, 3.0 M in THF, 82 mmol) dropwise. After 30 minutes, the mixture had formed a deep blue, sticky “ball.”

The 3-TR/TMS-imidazole mixture was cooled to −50° C. and, to this, was charged with the copper acetate/DMPU solution above via cannula. The residual sticky mass from the copper acetate/DMPU mixture was dissolved using DCM (50 mL) and transferred.

Methyl magnesium chloride (123.2 mL, 3.0 M solution in THF, 368 mmol) was added dropwise over 45 minutes to the combined reaction mixtures, which were then allowed to stir for 2 hours at −50° C. Subsequent HPLC analysis showed complete consumption of starting material. The mixture was allowed to warm to room temperature overnight, with stirring.

Toluene (800 mL) was added to the mixture, followed by a 5% acetic acid solution (600 mL). The aqueous layer was removed and discarded. The acetic acid wash was repeated. The organic layer was washed with brine (400 mL), 5% sodium bicarbonate solution (400 mL×2), followed by a brine wash (400 mL). The organic solution was dried over sodium sulfate, then concentrated to dryness under reduced pressure. The product was recovered as a viscous, light golden oil. Mass recovery was 146 grams (119% theoretical).

### Step 2 Compound 3 Preparation

Compound 2 (92 g, 202 mmol) and toluene (1000 mL, 10.9 vol) were charged to a reaction flask under nitrogen, and the solution was cooled to −10° C. A 32 wt % solution of peracetic acid in acetic acid (60 mL, 283 mmol, 1.4 eq) was added dropwise over about 30 min maintaining the temperature at −10° C. The reaction was held for approximately 20 h (HPLC showed 75% Cmpd 3, Cmpd 2 1.5%, 6% diastereomer; 5% epoxide). Starting at −10° C., a 20% aqueous sodium bisulfite solution (920 mL, 10 vol) was added carefully via the addition funnel, keeping the temperature below 10° C. Trifluoroacetic acid (16 mL, 202 mmol, 1 eq) was added, and the mixture was held for 3 h at 0-5° C. to complete desilylation (endpoint by HPLC). The lower aqueous layer was drained, and the organic layer was washed with a saturated solution of sodium bicarbonate (3×250 mL), followed by water (1×250 mL) and brine (1×150 mL). The organic layer was then dried over Na₂SO₄, filtered, and concentrated to a pasty solid (89 g). The residue was taken up in 1.5 vol of EtOAc and transferred to neat heptane (19 vol) to precipitate crude Cmpd 3 as an off-white solid (50 g, 62.5% yield; HPLC 79% Cmpd 3, 5.6% epoxide, 1.7% diastereomer). The crude Cmpd 3 (48.5 g) was triturated in hot acetonitrile (2 vol) at 60° C. for 4 h and then gradually cooled to ambient temperature overnight. The mixture was filtered using the recycled filtrate to rinse and wash the wet cake. After drying, the recovery was 64.3% (31.2 g; HPLC 93.5% Cmpd 3, 3.3% epoxide). To remove the epoxide impurity, the 31 Cmpd 3 was dissolved in DCM (250 mL, 8 vol), and a solution of 48% HBr in water was added (7.5 mL). The mixture was heated at 40° C. for 1 h (HPLC <0.3% epoxide). The mixture was cooled and transferred to a separatory funnel. The lower aqueous layer (brown) was removed, and the upper organic layer was washed with water (200 mL), saturated NaHCO₃ (150 mL), and brine (100 mL). The organic layer was dried over Na₂SO₄, filtered, and concentrated to a tan foam (32 g, —100% recovery). Methanol (64 mL, 2 vol) was added to the 32 g foam forming a slurry. To this was added a 1:1 solution of MeOH:water (60 mL, 2 vol) dropwise. The slurry cooled slightly below ambient temperature and filtered using recycled filtrate to rinse and wash the wet cake. The solids were dried to constant weight, affording 26.1 g Cmpd 3 (81% recovery; HPLC 97.8%). The overall yield for Step 2 was 32.5%.

### Step 3 VBP15 Preparation

Compound 3 (26 g, 65 mmol) and MeOH (156 mL, 6 vol) were mixed in a reaction flask and cooled to 0-5° C. A solution of K₂CO₃ (9.9 g, 72 mmol, 1.1 eq) in water (65 mL) was added dropwise, and the mixture was allowed to gradually warm to ambient temperature overnight. Analysis by HPLC showed 2.5 SM and another 5 mol % K₂CO₃ was added, and the mixture was stirred for another day (HPLC endpoint 1.1% Cmpd 3). The mixture was neutralized to pH 7 with 1.5 M HCl (53 mL), and ˜25% of the MeOH (30 g) was removed under vacuum to maximize recovery. After stirring for 2 days, the product was isolated by filtration using the recycled filtrate to transfer the wet cake to the funnel. The wet cake was dried under vacuum, affording 19.3 g VBP15 (83% yield) as an off-white powder. Analysis of the solids by HPLC showed 98.8% purity with 0.6% Cmpd 3 as the only major impurity.

Other uses of the disclosed methods will become apparent to those in the art based upon, inter alia, a review of this patent document.

What is claimed is:

1. A compound which is vamorolone Form II.

2. The compound of claim 1, further characterized by an X-ray powder diffraction pattern comprising peaks, in terms of ° 2θ, at about 11.8, about 15.9, and about 18.2 with radiation Cu Kα.

3. The compound of claim 1 or 2, further characterized by an X-ray powder diffraction pattern substantially as shown in FIG. 1 or 2.

4. A compound which is vamorolone Form III.

5. The compound of claim 4, further characterized by an X-ray powder diffraction pattern comprising peaks, in terms of ° 2θ, at about 19.4, about 25.8, and 29.3 with radiation Cu Kα.

6. The compound of claim 4 or 5, further characterized by an X-ray powder diffraction pattern substantially as shown in FIG. 3 or 4.

7. The compound of any one of claims 4 to 6, prepared by a process comprising the step of slowly concentrating by evaporation at room temperature a solution of vamorolone in a solvent and isolating the resulting crystals.

8. The compound of claim 7, wherein the solvent is dichloromethane or a mixture of dichloromethane/heptane.

9. A compound which is vamorolone Form IV.

10. The compound of claim 9, further characterized by an X-ray powder diffraction pattern comprising peaks, in terms of ° 2θ, at about 14.2, about 15.1, about 16.8, about 17.0, and about 24.8 with radiation Cu Kα.

11. The compound of claim 9 or 10, further characterized by an X-ray powder diffraction pattern substantially as shown in FIG. 5 or 6.

12. The compound of any one of claims 9 to 11, further characterized by a thermogravimetric analysis profile showing less than about 0.5% weight loss below about 200° C.

13. The compound of claim 12, further characterized by a thermogravimetric analysis profile substantially as shown in FIG. 7.

14. The compound of any one of claims 9 to 13, prepared by a process comprising the step of slowly concentrating by evaporation at room temperature a solution of vamorolone in a solvent and isolating the resulting crystals.

15. The compound of claim 14, wherein the solvent is acetone or a mixture of ethyl acetate/methanol.

16. A compound which is vamorolone Form V.

17. The compound of claim 16, further characterized by an X-ray powder diffraction pattern comprising peaks, in terms of ° 2θ, at about 12.1, about 14.8, about 15.0, and about 15.2 with radiation Cu Kα.

18. The compound of claim 16 or 17, further characterized by an X-ray powder diffraction pattern substantially as shown in FIG. 8.

19. The compound of any one of claims 16 to 18, further characterized by a thermogravimetric analysis profile showing less than about 0.5% weight loss below about 200° C.

20. The compound of claim 19, further characterized by a thermogravimetric analysis profile substantially as shown in FIG. 9.

21. The compound of any one of claims 16 to 20, further characterized by a melting event with an onset and peak temperatures of 234.3° C. and 242.7° C., respectively, as measured by differential scanning calorimetry.

22. The compound of claim 21, further characterized by a differential scanning calorimetry trace substantially as shown in FIG. 10.

23. The compound of any one of claims 16 to 22, prepared by a process comprising the step of slowly concentrating by evaporation at room temperature a solution of vamorolone in a solvent and isolating the resulting crystals.

24. The compound of claim 23, wherein the solvent is a mixture of ethyl acetate/dichloromethane.

25. A compound which is vamorolone Form VI.

26. The compound of claim 25, further characterized by an X-ray powder diffraction pattern comprising peaks, in terms of ° 2θ, at about 7.7, about 11.3, and about 11.5 with radiation Cu Kα.

27. The compound of claim 25 or 26, further characterized by an X-ray powder diffraction pattern substantially as shown in FIG. 11 or 12.

28. The compound of any one of claims 25 to 27, prepared by a process comprising the step of slowly concentrating by evaporation at room temperature a solution of vamorolone in a solvent and isolating the resulting crystals.

29. The compound of claim 28, wherein the solvent is a mixture of acetonitrile/ethyl acetate.

30. A compound which is vamorolone Form VII.

31. The compound of claim 30, further characterized by an X-ray powder diffraction pattern comprising peaks, in terms of ° 2θ, at about 18.4, about 28.2, about 29.1, and about 29.3 with radiation Cu Kα.

32. The compound of 30 or 31, further characterized by an X-ray powder diffraction pattern substantially as shown in FIG. 13.

33. The compound of any one of claims 30 to 32, further characterized by a thermogravimetric analysis profile showing less than about 1% weight loss below about 225° C.

34. The compound of claim 33, further characterized by a thermogravimetric analysis profile substantially as shown in FIG. 14.

35. The compound of any one of claims 30 to 34, further characterized by a melting event with an onset and peak temperatures of 227.0° C. and 236.9° C., respectively, as measured by differential scanning calorimetry.

36. The compound of claim 35, further characterized by a differential scanning calorimetry trace substantially as shown in FIG. 15.

37. The compound of any one of claims 30 to 36, prepared by a process comprising the step of slowly concentrating by evaporation at room temperature a solution of vamorolone in a solvent and isolating the resulting crystals.

38. The compound of claim 37, wherein the solvent is a mixture of acetonitrile/2-butanone, acetonitrile/dichloromethane, or dichloromethane/nitromethane.

39. A compound which is vamorolone Form VIII.

40. The compound of claim 39, further characterized by an X-ray powder diffraction pattern comprising peaks, in terms of ° 2θ, at about 16.1, and about 16.3 with radiation Cu Kα.

41. The compound of claim 39 or 40, further characterized by an X-ray powder diffraction pattern substantially as shown in FIG. 16 or 17.

42. The compound of any one of claims 39 to 41, further characterized by a thermogravimetric analysis profile showing less than about 0.5% weight loss below about 200° C.

43. The compound of claim 42, further characterized by a thermogravimetric analysis profile substantially as shown in FIG. 18.

44. The compound of any one of claims 39 to 43, further characterized by a melting event with an onset and peak temperatures of 226.8° C. and 237.7° C., respectively, as measured by differential scanning calorimetry.

45. The compound of claim 44, further characterized by a differential scanning calorimetry trace substantially as shown in FIG. 18.

46. The compound of any one of claims 39 to 45, prepared by a process comprising the step of slowly concentrating by evaporation at room temperature a solution of vamorolone in a solvent and isolating the resulting crystals.

47. The compound of claim 46, wherein the solvent is a mixture of acetonitrile/acetone, acetone/dichloromethane, ethyl acetate/tetrahydrofuran, methanol/2-propanol, or tetrahydrofuran/toluene.

48. The compound of any one of the preceding claims, wherein the compound has a chemical purity of at least about 95%, at least about 96%, at least about 97%, at least about 98%, at least about 99%, or at least about 99.5% by high performance liquid chromatography.

49. The compound of any one of the preceding claims, wherein the compound has total chemical impurities of not more than about 0.5% by high performance liquid chromatography.

50. The compound of any one of the preceding claims, wherein the compound has not more than about 0.5% by high performance liquid chromatography of an epoxide having the formula:

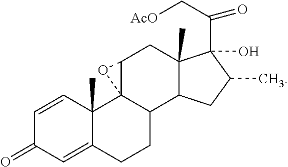

51. A pharmaceutical composition comprising at least one compound of any of the preceding claims and at least one pharmaceutically acceptable excipient.

52. The pharmaceutical composition of claim 51, wherein the compound is Form I vamorolone.

53. The pharmaceutical composition of claim 51 or 52, wherein the compound is administered orally.

54. The pharmaceutical composition of claim 53, wherein the compound is administered as a tablet or capsule.

55. The pharmaceutical composition of claim 53, wherein the compound is administered as a solution or suspension.

56. The pharmaceutical composition of claim 55, wherein the solution or suspension further comprises a flavoring agent.

57. A method of treating or reducing the symptoms of a disease having a significant and pathologic inflammatory component that can be addressed by inhibition of NF-κB muscular dystrophy, comprising the administration, to a patient in need thereof, of a therapeutically effective amount of a compound of any one of claims 1 to 50 or a pharmaceutical composition of any one of claims 51 to 56.

58. The method of claim 57, wherein the therapeutically effective amount is between 10 mg to 200 mg.

59. The method of claim 57, wherein the therapeutically effective amount is between 0.01 mg/kg to 10.0 mg/kg.

60. The method of claim 57, wherein the therapeutically effective amount is between 2 mg/kg to 6.0 mg/kg.

61. The method of any one of claims 57 to 60, wherein the disease is muscular dystrophy.

62. The method of claim 61, wherein the muscular dystrophy is chosen from Duchenne muscular dystrophy, Becker muscular dystrophy, limb girdle muscular dystrophy, congenital muscular dystrophy, facioscapulohumeral muscular dystrophy, myotonic muscular dystrophy, oculopharyngeal muscular dystrophy, distal muscular dystrophy, and Emery-Dreifuss muscular dystrophy.
